(19) United States
(12) Patent Application Publication
(10) Pub. No.: US 2007/0032452 A1
(43) Pub. Date: Feb. 8, 2007

(54) Blood soluble drag reducing hyaluronic acid

(76) Inventors: Kipling Thacker, Excelsior, MN (US); Marina Kameneva, Pittsburgh, PA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/364,577

(22) Filed: Feb. 27, 2006

Related U.S. Application Data
(60) Provisional application No. 60/657,119, filed on Feb. 28, 2005.

(51) Int. Cl. A61K 31/724

(52) U.S. Cl. 514/54

(57) ABSTRACT

The use of hyaluronic acid and physiologically acceptable salts thereof as drag reducing agents is described. The compositions of the invention can be used to increase aortic blood flow, increase arterial blood flow, increase venous blood flow, decrease blood pressure, decrease peripheral vascular resistance, diminish the development of atherosclerosis, and/or prevent lethality of hemorrhagic shock.

### CROSS REFERENCE TO RELATED APPLICATIONS

This application claims benefit under 35 U.S.C. § 119(e) to application U.S. Ser. No. 60/657,119, filed Feb. 28, 2005 (Attorney docket number 186327/US), entitled “Blood Soluble Drag Reducing Hyaluronic Acid”, the contents of which are incorporated herein by reference in their entirety for all purposes.

### FIELD OF THE INVENTION

The present invention relates to improved microflow drag reducing polymers for use in blood as well as the restoration and/or enhancement of microcirculation and tissue oxygenation. The invention is further directed to methods for the restoration and/or enhancement of microcirculation and perfusion and oxygenation of mammalian tissues due to changes in fluid properties of blood induced by drag reducing polymers provided herein.

### BACKGROUND OF THE INVENTION

Drag reducing polymers (DRPs) provide positive hemodynamic effects in various acute and chronic animal models. Nanomolar concentrations of various DRPs that are injected intravenously have been shown to increase aortic and arterial blood flow and decrease blood pressure and peripheral vascular resistance. Intravenous injections of DRPs have also been shown to diminish the development of atherosclerosis in atherogenic animal models.

The DRPs that have been studied thus far have been polyacrylamides, polyethylene oxides, polyethylene glycols, a polysaccharide extracted from okra and calf thymus DNA. Each of these materials has had one or more disadvantages, such as presence of toxicity or mechanical degradation upon application of stress to a solution of the selected material

Therefore, a need exists for the identification of an endogenous material of a mammal that can be used as a DRP, at an increased concentration greater than that found in the natural state of the mammal, such that the DRP can provide one of more beneficial effects to the mammal.

### BRIEF SUMMARY OF THE INVENTION

The present invention provides an endogenously derived DRP that can be used to increase aortic blood flow, arterial blood flow, increase capillary blood flow, increase venous blood blow, decrease blood pressure, decrease peripheral vascular resistance, diminish the development of atherosclerosis, and/or prevent lethality of hemorrhagic shock. Suitable DRPs of the present invention include hyaluronic acid and hyaluronic acid derivatives, such as pharmaceutically acceptable salts of hyaluronic acid.

The present invention further provides suitable pharmaceutical compositions of the DRPs of the invention.

Additionally, the present invention also provide packaged pharmaceutical formulations that contain the DRPs of the invention and instructions how to use the DRP(s).

The DRPs of the invention generally have molecular weights of from about 500 kD to about 7,000 kD, more particularly between about 500 kD and about 2000 kD, e.g., between about 680 kD to about 1500 kD. Useful concentrations of the DRPs are between about 0.1 ppm and about 1000 ppm.

### BRIEF DESCRIPTION OF THE DRAWINGS

FIG. 1 demonstrates that suitable concentrations of low molecular weight sodium hyaluronate are effective in reducing the flow resistance in an aqueous solution simulating behavior in blood.

FIG. 2 shows a higher drag-reducing efficiency of HA with MW of ˜1500 kD compared to that of PEO with MW 2000 kD, and an incredibly low rate of mechanical degradation of this HA which was circulating in the in vitro flow system at a flow rate of 4.5 L/min at the concentration in the solution of 100 ppm.

FIG. 3 shows a higher drag-reducing efficiency of HA with MW of ˜1500 kD compared to that of PEO with MW 2000 kD, and an incredibly low rate of mechanical degradation of this HA which was circulating in the in vitro flow system at a flow rate of 4.5 L/min at the concentration in the solution of 100 ppm.

FIG. 4 shows viscosity results for the HA-1500 and PEO-2000 solutions at the concentrations of 2500, 1000 and 500 ppm @ 24.5° C. presented as the mean and standard deviations of three measurements.

FIG. 5 enlarges FIG. 4.

FIG. 6 depicts data on elasticity of PEO-2000 solutions.

FIG. 7 shows data from FIG. 6 in a larger scale.

FIG. 8 shows the results of several tests with turbulent flow of the HA-1500 and PEO-2000 in a pipe.

FIG. 9 depicts data represented in Tables 2 and 3.

FIG. 10 shows results of microchannel studies.

FIG. 11 depicts an image of a control sample of bovine RBC suspension (23% Ht in PBS/albumin solution) flow (flow rate=0.1 ml/min) in a microchannel.

FIG. 12 depicts an image of a 10 ppm HA 1481 kDa sample in bovine RBC suspension in a microchannel.

FIG. 13 depicts an image of a 50 ppm HA 1481 kDa sample in a bovine RBC suspension in a microchannel.

FIG. 14 depicts an image of a 10 ppm PEO-2000 sample in a bovine RBC suspension in a microchannel.

FIG. 15 depicts an image of a 50 ppm PEO-2000 sample in a bovine RBC suspension in a microchannel.

FIG. 16 provides Brookfield rheometer results for solutions of HA-990 and PEO-1000 at the concentrations of 2500, 1000, and 500 ppm measured @ 24.3° C.

FIG. 17 shows the results of FIG. 16 excluding the 2500 ppm HA preparation.

FIG. 18 shows viscosity results for the HA and PEO solutions at the concentrations of 2500, 1000 and 500 ppm @ 24.5° C. presented as the mean and standard deviations of three measurements.

FIG. 19 provides the results as in FIG. 18 excluding the 2500 ppm HA preparation.

FIG. 20 provides elasticity of HA-990 2500 ppm solution.

FIG. 21 shows the results of several tests with turbulent flow of the HA-990 and PEO-1000 in a pipe.

FIG. 22 depicts data represented in Tables 5 and 6.

FIG. 23 depicts data represented in Tables 5 and 6.

FIG. 24 depicts an image of a control sample of bovine RBC suspension (23% Ht in PBS/albumin solution) flow (flow rate=0.1 ml/min) in a micro channel.

FIG. 25 depicts an image of a 10 ppm PEO-1000 sample in bovine RBC suspension in a microchannel.

FIG. 26 depicts an image of a 10 ppm 9863 kDA HA sample in bovine RBC suspension in a microchannel.

FIG. 27 depicts an image of a 10 ppm PEO WSR-301 sample in bovine RBC Suspension in a microchannel.

### DETAILED DESCRIPTION

The present invention provides a unique and unexpected advantage that increased concentrations of an endogenously occurring material, relative to the naturally occurring level of such an endogenous material, can be used as a drag reducing polymer (DRP). Surprisingly, such an endogenous material is hyaluronic acid (or physiologically acceptable salts thereof), hereinafter referred to as “HA”. HA provides the unique advantage that the physiology of the mammal that requires treatment can more readily accept an endogenous substance rather than a foreign material, such as a polyethylene oxide.

The term hyaluronic acid is known in the art and it should be understood, that the term “hyaluronic acid” includes hyaluronan. Hyaluronic acid, under physiological conditions, is converted into various forms, based on electrolytes and other physiological medium. Therefore, it should be understood that once the hyaluronic acid is placed in an electrolytic solution, it is more correctly known as hyaluronan.

HA is a carboxyl containing polysaccharide. Carboxyl containing polysaccharides useful to treat the various diseases or conditions identified throughout the application are considered within the scope fo the present invention.

The term “a carboxyl-containing polysaccharide” is intended to mean a polysaccharide containing at least one carboxyl group. The polysaccharide chosen may initially contain carboxyl groups or it may be derivatized to contain carboxyl groups. Examples of carboxyl-containing polysaccharides include, but are not limited to, carboxymethyl cellulose, carboxymethyl chitin, carboxymethyl chitosan, carboxymethyl starch, alginic acid, pectin, carboxymethyl dextran, and glucosaminoglycans such as heparin, heparin sulfate, chondroitin sulfate and hyaluronic acid (HA). The most preferred carboxyl-containing polysaccharides are carboxymethyl cellulose, carboxymethyl chitin and HA. The most preferred carboxyl-containing polysaccharide is HA.

The compositions of the invention include a carboxyl-containing polysaccharide, or alternatively, a pharmacologically acceptable salt of the polysaccharide can be used, e.g., hyaluronan. Suitable pharmacologically acceptable salts are alkali or alkaline earth metal salts. Therefore, in one embodiment, the composition contains sodium hyaluronate.

Carboxyl-containing polysaccharides that can be used to prepare useful compositions of the invention are known compounds that are described, for example, in U.S. Pat. No. 4,517,295 and U.S. Pat. No. 4,141,973; and Handbook of Water Soluble Gums and Resins, Chapter 4, by Stelzer & Klug, published by McGraw-Hill, 1980. Processes for preparing the carboxyl-containing polysaccharide, HA, are illustrated in the Balazs patent, which details a procedure for extracting HA from rooster combs, and in U.S. Pat. No. 4,517,295 that describes fermentation process for making HA. The HA used to make the DRP should be highly purified (medical grade quality) for in vivo applications.

The phrase “physiologically acceptable salts thereof (of hyaluronic acid)” is intended to include those derivatives wherein one or more of the acidic protons of the carboxylic acid groups of the hyaluronic acid moiety is substituted by a counterion. Suitable counterions include groups I, II, III and IV metals, ammonium complexes, amino acid complexes, etc. For example, the physiologically acceptable salt can include sodium, lithium, magnesium, potassium, ammonium ion and various amino acids as counterions.

The HA generally has a molecular weight of from low molecular weight oligosaccharides of HA to about 7,000 kD, in particular between about 500 kD and about 2000 kD, more particularly between about 600 kD and 1700 kD and in one embodiment about 1100 kD. In one embodiment, the HA is a sodium salt.

HA can be provided in the form of a pharmaceutical composition. In one particular aspect, the pharmaceutical composition can be in the form of an injectable intravenous preparation. In another aspect, the composition can be placed into an intravenous solution that is administered over a period of time, e.g., an iv drip.

The pharmaceutical composition can be an aqueous solution that includes sodium salt(s), i.e., sodium chloride, potassium salt(s), i.e., potassium chloride, calcium salt(s), i.e., calcium chloride, magnesium salt(s), such as magnesium chloride, sodium acetate, sodium citrate and/or sodium phosphate. Alternatively, the solution can be a saline solution or PBS.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the HA may be dried by any art-known technique, such as lyophilization, and reconstituted prior to use.

For prolonged delivery, the HA can be formulated as a depot preparation for administration by implantation, intravenous or intraperitoneal injection. The HA may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the HA for percutaneous absorption may be used. To this end, permeation enhancers may be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

The pharmaceutical compositions may, if desired, be presented in a pack or dispenser device which may contain one or more unit dosage forms containing the HA. The pack may, for example, comprise metal or plastic foil, such as a blister pack. The pack or dispenser device may be accompanied by instructions for administration.

The HA and pharmaceutical compositions described herein can be administered to achieve the intended result, for example in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of HA to a patient suffering from bleeding due to wound, trauma or surgery ameliorates the effect of the loss of blood, possibly by improved peripheral tissue oxygenation and better waste removal from peripheral tissue. Therapeutic benefit also includes halting or slowing the progression of the disease, regardless of whether improvement is realized

For prophylactic administration, the HA may be administered to a patient at risk of developing one of the previously described diseases. Alternatively, prophylactic administration may be applied to avoid the onset of symptoms in a patient diagnosed with the underlying disorder.

The amount of HA administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Effective dosages may be estimated initially from in vitro assays. For example, an initial dosage for use in animals may be formulated to achieve a circulating blood or serum concentration of HA that is at or above an IC₅₀ of the HA as measured in as in vitro assay, such as those described in Kameneva, cited herein below, and those references cited therein. Calculating dosages to achieve such circulating blood or serum concentrations taking into account the bioavailability of the HA is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pagamonon Press, and the references cited therein.

Initial dosages can also be estimated from in vivo data, such as animal models. Animal models useful for testing the efficacy of compounds to treat or prevent the various diseases described above are well-known in the art.

Dosage amounts of the HA will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma levels of the HA which are sufficient to maintain therapeutic or prophylactic effect. For example, the HA may be administered once per week, several times per week (e.g., every other day), once per day or multiple times per day, depending upon, among other things, the mode of administration, the specific indication being treated and the judgment of the prescribing physician.

Preferably, the HA will provide therapeutic or prophylactic benefit without causing substantial toxicity. Toxicity of the HA may be determined using standard pharmaceutical procedures. The dose ratio between toxic and therapeutic (or prophylactic) effect is the therapeutic index.

The DRPs of the present invention can be used to increase aortic blood flow, increase arterial blood flow, increase venous blood flow, decrease blood pressure, decrease peripheral vascular resistance, diminish the development of atherosclerosis, and/or prevent lethality of hemorrhagic shock. The DRPs are provided in therapeutically effective amounts.

The DRPs of the invention can be evaluated using standard methods in the art to determine the efficacy in the increase of aortic blood flow, increase arterial blood flow, increase capillary blood flow, increase venous blow flow, decrease blood pressure, decrease peripheral vascular resistance, diminish the development of atherosclerosis, and/or prevent lethality of hemorrhagic shock. Suitable animal models are known as described by Kameneva et al. “Blood soluble drag-reducing polymers prevent lethality from hemorrhagic shock in acute animal experiments”, Biorheology 41 (2004), 53-64, the contents of which are incorporated herein in their entirety, including the teachings of those references cited therein.

### EXAMPLES

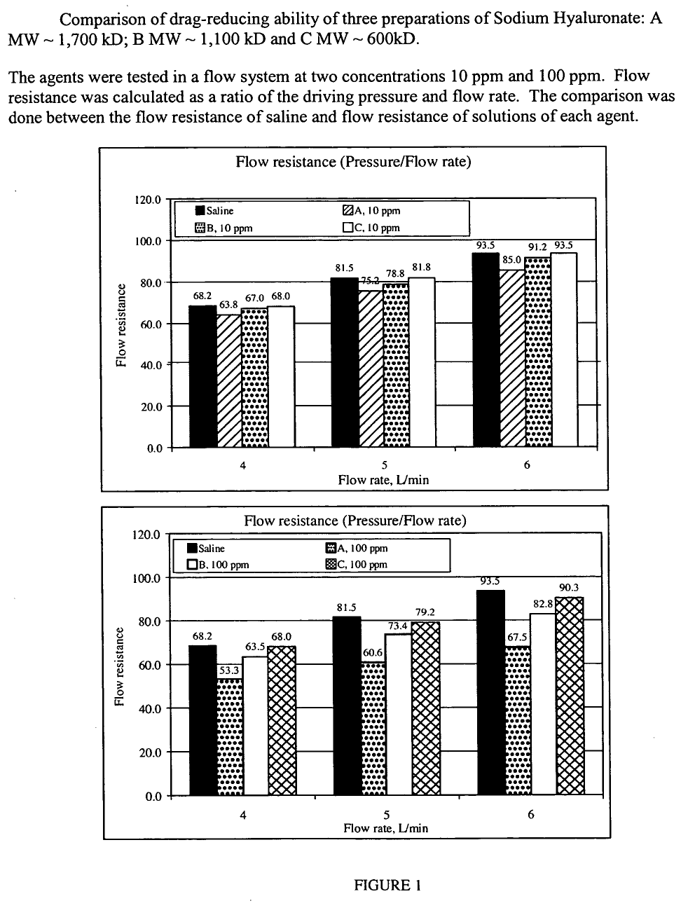

FIG. 1 provides evidence that hyaluronate reduces hydrodynamic resistance to turbulent flow of an aqueous solution.

Study of Rheological and Drag-Reducing Properties of Sodium Hyaluronate (NaHy) with MW of 1,481,000 Da (HA-1500), Compared to Polyethylene Oxide with Molecular Weight 2,000,000 Da (PEO-2000)

This study compared rheological, microrheological and drag-reducing properties of sodium hyaluronate (Lifecore Biomedical, Inc., Minnesota) with MW=˜1,500 kDa (HA-1500) and polyethylene oxide with MW of 2000 kDa (PEO-2000).

I. Molecular Characteristics

Molecular characteristics of the HA-1500 polymer obtained by Viscotek GPC analysis. For all of the GPC tests the solvent was 0.1M NaNO3 with 0.01% NaN3. The flow rate was 0.5 ml/min and the temperature was 30° C.

Viscosity Average Molecular Weight, Mv: 1,262,000 Da

Weight Average Molecular Weight, Mw: 1,298,000 Da

Intrinsic Viscosity, IV: 27.2 dL/g

Hydrodynamic Radius, Rh: 82 nm

Radius of Gyration, Rg: 106 nm

PDI: 1.1

Molecular characteristics of the PEO-2000 obtained from Viscotek GPC analysis: For all of the GPC tests the solvent was 0.1M NaNO3 with 0.01% NaN3. The flow rate was 0.5 ml/min and the temperature was 30° C.

Mv: 2,008,000 Da

Mw: 2,385,000 Da

Intrinsic Viscosity, IV: 8.9 dL/g

Rh: 66 nm

Rg: 85 nm

PDI: 2.4

II. Rheological Evaluation:

HA-1500 and PEO-2000 solution viscosity was evaluated using several types of rheometers/viscometers. Original HA-1500 solution (0.97% concentration) was diluted with saline to achieve the polymer concentrations of 2500, 1000, 100 and 10 parts per million (ppm). Commercial PEO with MW of 2000 kD (Aldrich Chemical Co, #37,280-3) was used for this study. The stock PEO-2000 solution was prepared at the concentration of 2500 ppm.

1. Capillary Viscometry.

Results obtained @ 23° C. are given in Table 1.

TABLE 1

| Concentration | Sodium Hyaluronate (KDA 1481) | PEO-1000 Viscosity |
|---|---|---|
| (ppm) | Viscosity (cP) | (cP) |
| 10 | 1.02 | 1.10 |
| 100 | 1.33 | 1.22 |
| 500 | 3.57 | 1.64 |
| 1000 | 8.7 @ 166 s⁻¹ | 2.4 |
| 2500 | 77.6 @ 37 s⁻¹ | 5.7 @ 239 s⁻¹ |

Since capillary viscometers do not allow varying shear rates, and, in each case, the shear rate value depends on the flow rate of the tested solution in the capillary (i.e. on viscosity and diameter of the capillary tube), viscosity of HA-1500 at the concentrations of 1000 and 2500 ppm and PEO-2000 at the concentration of 2500 ppm are reported along with the shear rates calculated based on the measurement results and specific viscometer parameters. If the solution viscosity is below 4 cP, the shear rates in the capillary viscometers exceed 300 s⁻¹ and at these shear rates the polymer solution viscosity reaches its asymptotic value and does not have dependency on shear rate anymore. Thus, viscosities of HA at the concentrations 500 ppm and below and PEO at the concentrations 1000 ppm and below are given in the Table with no shear rate values.

2. Rotational Viscometry.

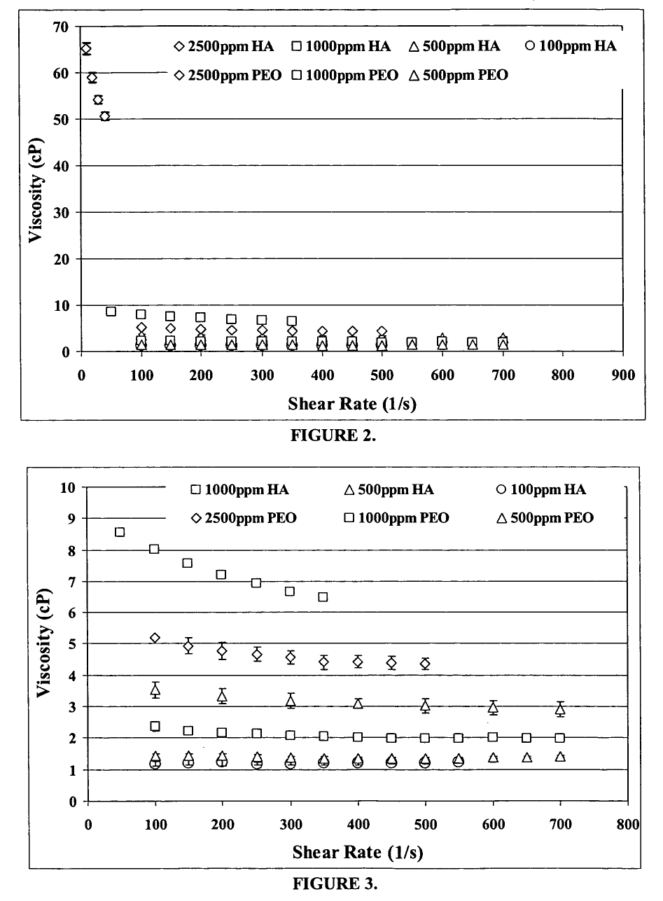

Brookfield rheometer measurement results for the solutions of HA-1500 and PEO-2000 at the concentrations of 2500, 1000, and 500 ppm tested @ 24° C. are shown in FIG. 2. Data represent mean and standard deviations of three measurements. The Brookfield rheometer at the current set up did not allow viscosity of the HA-1500 solution at the concentration of 2500 ppm to be measured at shear rates above 50 s⁻¹.

The same concentration of 2500 ppm, viscosity of the HA-1500 solution is about 10 times higher than viscosity of the PEO-2000 solution and more than 2 times higher than the viscosity of HA-1000 which was 21 cP at the same shear rate of 50 s⁻¹. FIG. 3 shows the same results excluding the 2500 ppm HA preparation to increase the scale.

3. Viscosity Measured in a Capillary Oscillating Flow (Vilastic).

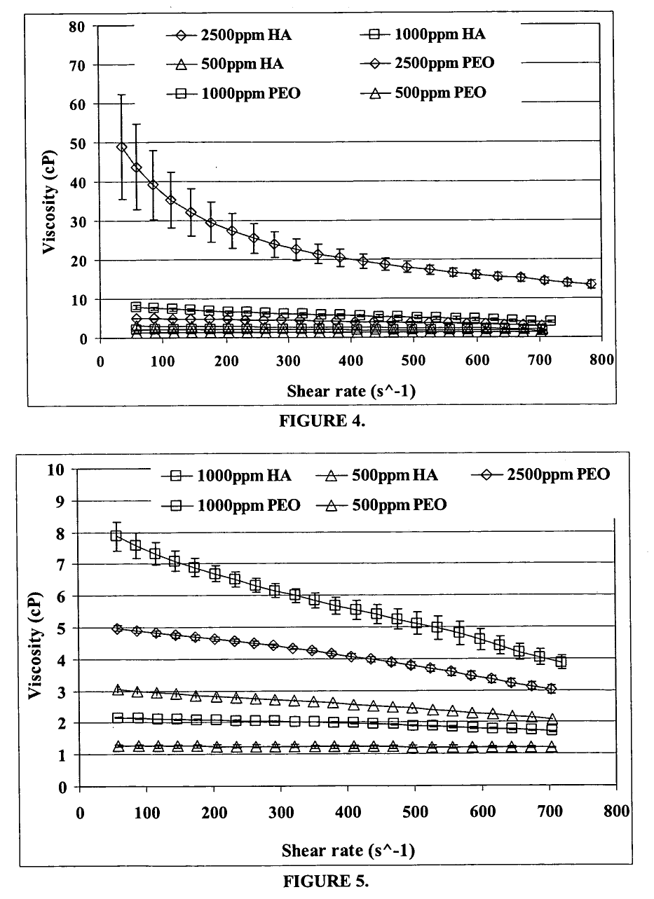

Viscosity data obtained using the Vilastic rheometer are presented in FIG. 4.

FIG. 4 shows viscosity results for the HA-1500 and PEO-2000 solutions at the concentrations of 2500, 1000 and 500 ppm @ 24.5° C. presented as the mean and standard deviations of three measurements. Once again, as was shown using Brookfield rheometer, viscosity of HA-1500 solution at the concentration of 2500 ppm is much higher than viscosity of the PEO-2000 solution (˜9 times at the shear rate ˜60 s⁻¹).

FIG. 5 enlarges FIG. 4 excluding the 2500 ppm HA-1500 preparation to increase the scale.

As seen in FIGS. 2 through 5, the HA-1500 demonstrates a non-Newtonian behavior at the all tested concentrations. At high shear rates, viscosities were found to be the same for HA-1500 at C=500 ppm and for PEO-2000 at C=1000 ppm. PEO-2000 demonstrates a pure Newtonian behavior (independence of viscosity on shear rate) at the concentrations of 500 ppm.

4. Elasticity Tests.

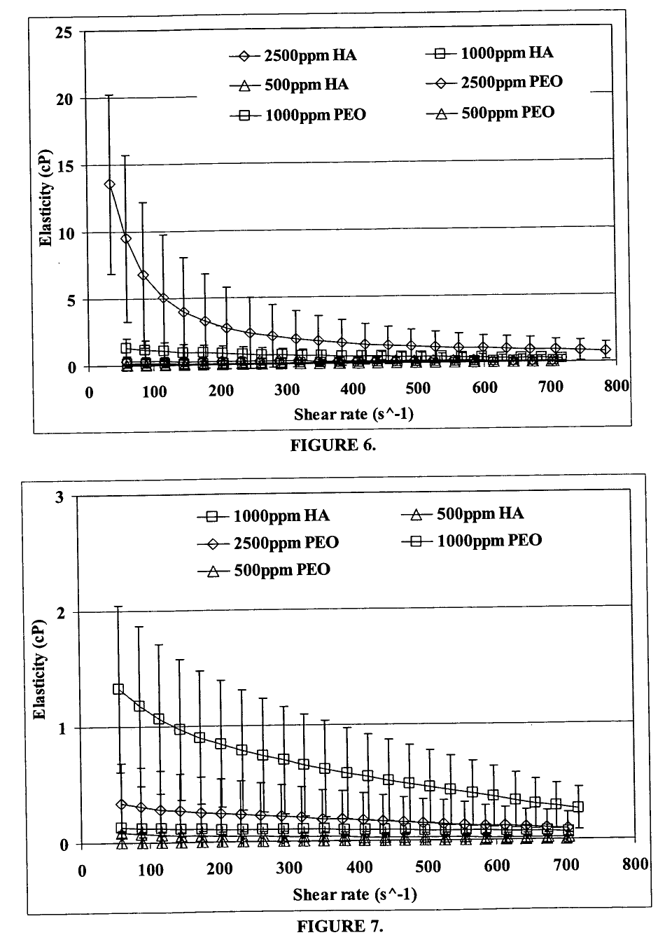

Elasticity of HA-1500 solutions at the concentrations of 2500 and 1000 ppm measured by Vilastic was found to be highly dependant on shear rates providing additional evidence of a strong non-Newtonian behavior of these solutions (See FIGS. 6 and 7). Data on elasticity of PEO-2000 solutions are shown in FIG. 6 for a comparison demonstrating much slighter non-Newtonian behavior of this polymer at the highest tested concentration (2500 ppm).

FIG. 7 shows the results in a larger scale. While HA-1500 solution prepared at the 1000 ppm concentration demonstrates a significant increase in the low shear elasticity, the PEO-2000 solution at the same concentration has much lower elasticity at low shear rates. Elasticity of both polymer solutions at the concentrations of 500 ppm was found to be negligible.

III. Hydrodynamic Evaluation.

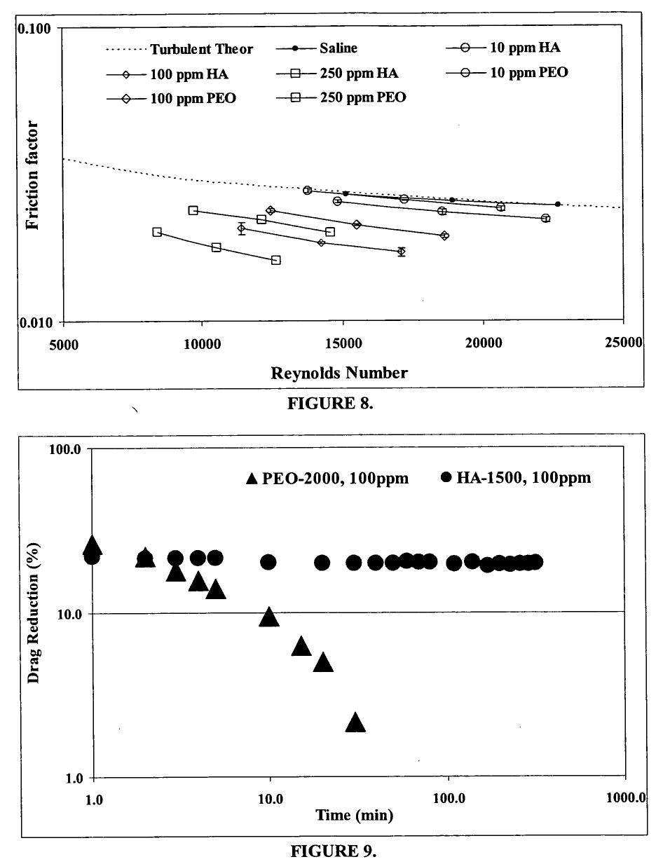

FIG. 8 shows the results of several tests with turbulent flow of the HA-1500 and PEO-2000 in a pipe. The plot shows friction factor (Lambda) versus Reynolds number.

As seen in FIG. 8, at concentrations of 10 ppm flow resistance of the HA-1500 is much lower than that of saline while flow characteristics of PEO-2000 solutions are almost exactly the same as those of saline. At both 100 ppm and 250 ppm concentrations HA-1500 demonstrates higher drag-reducing ability than PEO-2000 at the studied flow rates (37% vs. 25% maximum drag reduction at 100 ppm and 43% vs. 27% maximum drag reduction at 250 ppm respectively). Since the viscosity of HA-1500 is much higher than that of PEO-2000, Reynolds numbers corresponding to the same flow rates are much lower for the HA solutions. Therefore, it is not possible to directly compare flow resistance of these polymer solutions at the same flow rate. However, at the same Reynolds numbers the Friction Factors of HA-1500 solutions are about 20% lower that those of PEO-2000 at 100 ppm and up to 40% lower than PEO-2000 at 250 ppm. In each test, during the second run at the same flow rates PEO-2000 showed much lower drag-reducing effect than during the first run due to mechanical degradation, while HA-1500 demonstrated exactly the same drag-reducing effect during both runs.

IV. Mechanical Degradation Study.

It was previously found that polyethylene oxide while being one of the best drag reducers is extremely fragile and loses its drag-reducing ability when exposed to shear stress due to mechanical degradation. The mechanical degradation of the HA-1500 was tested in the circulating loop at constant wall shear stress of 45 N/m² (flow rate ˜4.5 L/min) at the solution concentration of 100 ppm. The HA-1500 polymer was found to be resistant to mechanical degradation with no loss of drag-reducing activity over the five hour test period. PEO-2000 at the solution concentration of 100 ppm tested at the same wall shear stress conditions completely lost its drag-reducing ability in 30 min.

V. Microflow Channel Study of the HA-1500 Effect on the Laminar Flow of Red Blood Cell (RBC) Suspensions.

Previous studies demonstrated that drag-reducing polymers added to a RBC suspension flowing in a microchannel with a diameter less that 0.5 mm redistribute RBCs across the microchannel and attenuate the “plasma-skimming” effect. Attenuation of near-wall plasma layer in a channel with rigid walls is accompanied by an increase in driving pressure/wall shear stress. Thus a comparison of driving pressure vs. flow rate for RBC suspensions with and without DRPs characterizes microhemodynamic properties of polymer preparations.

Farm fresh bovine RBCs were washed with PBS and resuspended in a PBS/albumin solution at a hematocrit of 19%. RBC shape was verified using light microscopy. The effects of HA-1500 and PEO-2000 additives on the flow of RBCs in a straight microchannel in laminar flow was studied using a circulating system consisting of a syringe pump, a pressure transducer, and a capillary tube with a diameter of 410 mm and length of 12.7 cm. The RBC suspension in the syringe was kept well mixed using a small magnetic stirring bar placed inside the syringe and agitated by a magnet from outside, and the capillary was oriented in a vertical position in order to prevent RBC sedimentation on the walls. Flow rates were varied from 0.5 to 2 ml/min. Pressure vs. flow relationships were measured for each polymer at each concentration (10 ppm and 50 ppm), normalized to account for pressure change due to viscosity alone, and compared to the control RBC suspension in PBS with no polymer added. In such settings, an increase in pressure compared to control would indicate that the RBCs moved closer to the channel walls and near wall viscosity increased.

The results of these studies are shown in Tables 2 and 3 and FIG. 9. Table 2 shows the percentage of changes in the driving pressure for each flow rate after addition of HA-1500 and PEO-2000 to the RBC suspension. Both polymers produced an increase in driving pressure at both concentrations.

TABLE 2
% Increase in Pressure

| | Control | HA | HA | PEO | PEO |
|---|---|---|---|---|---|
| Flow Rate | P | 10 ppm P | 50 ppm P | 50 ppm P | 10 ppm |
| (mL/min) | (mmHg) | (mmHg) | (mmHg) | (mmHg) | (mmHg) |
| 0.5 | 0.0 | 13.2 | 13.0 | 11.1 | 9.8 |
| 1 | 0.0 | 9.9 | 10.8 | 7.2 | 6.6 |
| 1.5 | 0.0 | 7.0 | 8.7 | 6.4 | 4.6 |
| 2 | 0.0 | 7.5 | 9.7 | 7.5 | 4.5 |

Table 3 presents p-values for all of the experiments to test statistical significance of the difference with controls. Both polymers at both tested concentrations produced a statistically significant increase in the driving pressure after addition of the polymer to the flowing suspension of RBCs.

TABLE 3
tTEST

| Flow Rate | | | | |
|---|---|---|---|---|
| (mL/min) | HA 10 ppm | HA 50 ppm | PEO 50 ppm | PEO 10 ppm |
| 0.5 | 0.0001 | 0.0000 | 0.0000 | 0.0022 |
| 1 | 0.0000 | 0.0000 | 0.0104 | 0.0001 |
| 1.5 | 0.0000 | 0.0000 | 0.0038 | 0.0002 |
| 2 | 0.0000 | 0.0000 | 0.0357 | 0.0000 |

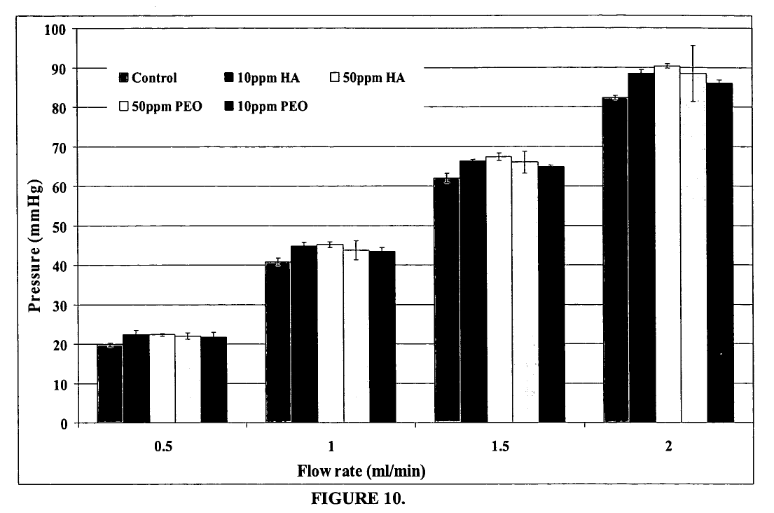

FIG. 10 shows the actual results of the microchannel studies. There is a slight but statistically significant difference between flow of RBC suspensions with and without addition of HA-1500 or PEO-2000 at the all studied flow rates.

VI. Microscopic Microchannel Studied of the Effects of Polymer Additives on RBC Distribution Across the Channel

Microscopic studies of bovine RBC suspension (23% Ht in PBS/albumin solution) flow (flow rate=0.1 ml/min) were performed in a microchannel with dimensions: 200 mm×80 mm×50 mm main channel, 50 mm×80 mm×20 mm branch.

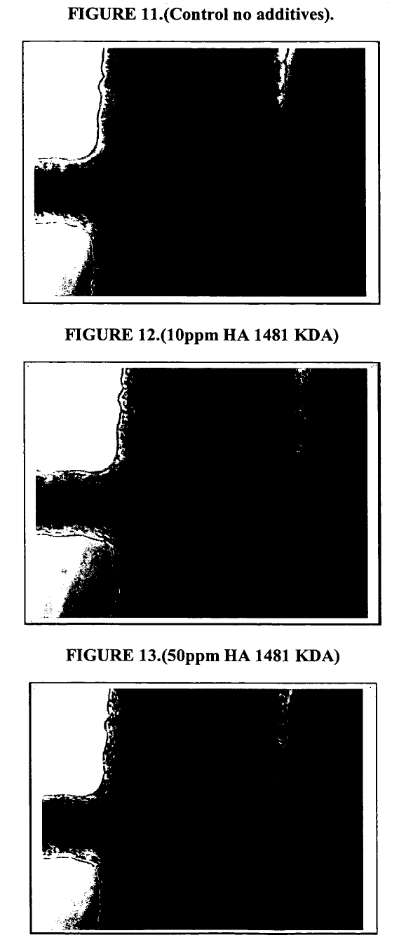

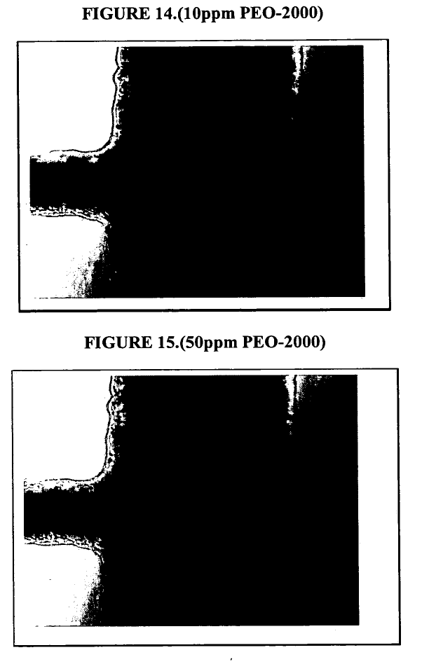

FIGS. 11 through 15 show images obtained in these tests with a 10 or 50 ppm concentration of the additives specified. Development of the near-wall plasma layer is clearly seen along with the red blood cells moving to the wall after addition of the polymers.

Only control RBC suspension with no DRP additives demonstrates a cell-free near-wall plasma layer. Both HA-1500 and PEO-2000 induced a considerable increase in the near-wall concentration of RBCs.

The data obtained demonstrated that the HA-1500 additive had much higher drag-reduction efficiency at all tested concentrations than PEO-2000. HA-1500 was found extremely stable against mechanical stress. No mechanical degradation was observed after 320 minutes of exposure to high flow shear stress which caused very fast total degradation of PEO-2000 (within 30 min). Viscosity and elasticity of HA-1500 were found to be much higher that those of PEO-2000 at the same concentrations. In the capillary tube with diameter of 410 μm both polymers showed a statistically significant increase in driving pressure which signified that even in this size of microchannels where a near-wall plasma layer was not well developed, these two polymers, HA-1500 and PEO-2000, changed distribution of RBCs across the tube at the studied concentrations. Both polymers showed the ability to affect flow of RBC suspension in a smaller microchannel (200 μm diameter) and thus the ability to change RBC distribution in microvessels. HA-1500 demonstrated a slightly higher effect on the RBC flow behavior in this microchannel than PEO-2000. Based on in vitro studies, HAO has the potential to be efficient in improvement of microcirculation in animal models of hypoperfusion and other blood flow pathologies.

Study of Rheological and Drag-Reducing Properties of Sodium Hyaluronate (NaHy) with MW of 986,300 Da (HA-990), Compared to Polyethylene Oxide with Molecular Weight 1,000,000 Da

This study compared rheological and drag-reducing properties of sodium hyaluronate MW 986,300 (HA-990) (Lifecore Biomedical, Inc., Minnesota, USA) and polyethylene oxide with MW of 1000 kD (PEO-1000).

I. Molecular Characteristics

Molecular characteristics of the HA-990 polymer obtained by Viscotek GPC analysis. For all of the GPC tests the solvent was 0.1M NaNO3 with 0.01% NaN3. The flow rate was 0.5 ml/min and the temperature was 30° C.

Viscosity Average Molecular Weight, Mv: 922,000 Da

Weight Average Molecular Weight, Mw: 1,111,000 Da

Intrinsic Viscosity, IVw: 20.4 dL/g

Hydrodynamic Radius, Rh: 67.8 nm

Radius of Gyration, Rg: 80.2 nm

PDI: 1.9

Molecular characteristics of the PEO-1000 from Viscotek GPC analysis. For all of the GPC tests the solvent was 0.1M NaNO3 with 0.01% NaN3. The flow rate was 0.5 ml/min and the temperature was 30° C.

Mv: 1,044,000 Da

Mw: 1,113,000 Da

Rh: 44.5 nm

Rg: 57.9 nm

Ivn: 3.5 dL/g

II. Rheological Evaluation:

HA-990 and PEO-1000 solution viscosity was evaluated using several types of rheometers/viscometers. Original HA-990 solution (0.95% concentration) was diluted with saline to achieve the polymer concentrations of 2500, 1000, 500, 100 and 10 parts per million (ppm). Commercial PEO with MW of 1000 kD (Aldrich Chemical Co, #37,278-1) was used for this study. The stock PEO-1000 solution was prepared at the concentration of 2500 ppm.

1. Capillary Viscometry.

Results obtained @ 21° C. are given in Table 4.

TABLE 4

| Concentration | Sodium Hyaluronate (KDA 9863) | PEO-1000 Viscosity |
|---|---|---|
| (ppm) | Viscosity (cP) | (cP) |
| 10 | 1.09 | 1.04 |
| 100 | 1.27 | 1.10 |
| 500 | 2.9 | 1.4 |
| 1000 | 4.75 @ 237 s⁻¹ | 1.91 |
| 2500 | 26.4 @ 66 s⁻¹ | 3.78 @ 297 s⁻¹ |

Since capillary viscometers do not allow varying shear rates, and, in each case, the shear rate value depends on the flow rate of the tested solution in the capillary (i.e. on viscosity and diameter of the capillary tube), viscosity of HA-990 at the concentrations of 1000 and 2500 ppm and PEO-1000 at the concentration of 2500 ppm are reported along with the shear rates calculated based on the measurement results and specific viscometer parameters. If the solution viscosity is below 3 cP, the shear rates in the capillary viscometers exceed 300 s⁻¹ and at these shear rates the polymer solution viscosity reaches its asymptotic level and does not have dependency on shear rate anymore. Therefore, viscosities of HA and PEO at the concentrations 500 ppm and below are given in the Table with no shear rate values.

2. Rotational Viscometry.

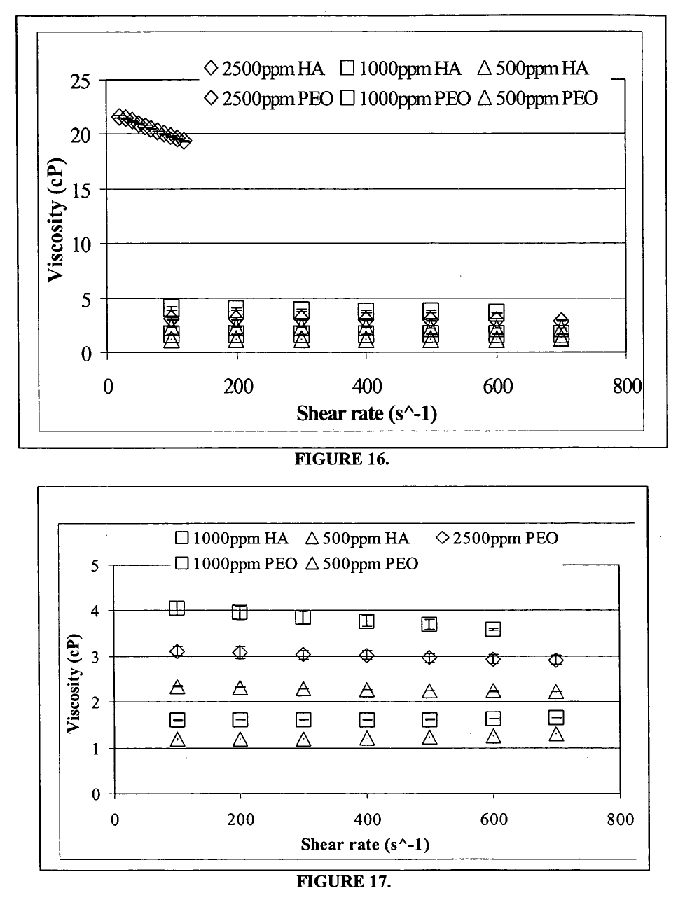

Brookfield rheometer results for the solutions of HA-990 and PEO-1000 at the concentrations of 2500, 1000, and 500 ppm measured @ 24.3° C. are shown in FIG. 16. Data represent mean and standard deviations of three measurements. The Brookfield rheometer did not allow for measuring HA-990 solution viscosity at the concentration of 2500 ppm at shear rates above 120 s⁻¹.

At the same concentration of 2500 ppm, viscosity of the HA-990 solution is more than six times higher than viscosity of the PEO-1000 solution (both measured at the same shear rate of 99.99 s-1). FIG. 17 shows the same results excluding the 2500 ppm HA preparation to reduce the scale.

3. Viscoelasticity.

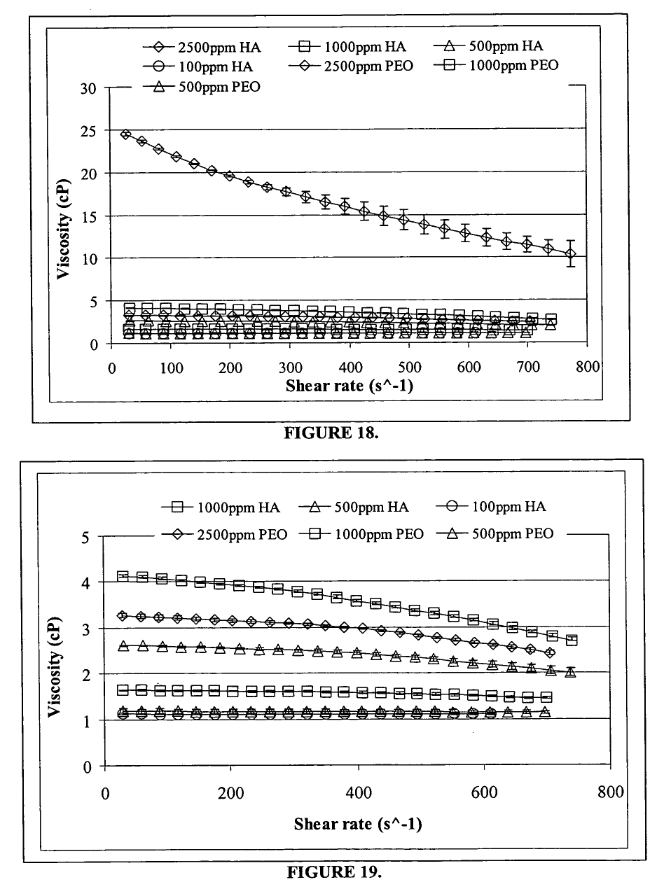

Viscosity data obtained using the Vilastic rheometer are presented in FIG. 18.

FIG. 18 shows viscosity results for the HA and PEO solutions at the concentrations of 2500, 1000 and 500 ppm @ 24.5° C. presented as the mean and standard deviations of three measurements. Once again, as was shown using a Brookfield rheometer, viscosity of HA-990 solution at the concentration of 2500 ppm is much higher than viscosity of the PEO-1000 solution (6.7 times at the shear rate ˜100 s⁻¹).

FIG. 19 shows the same results as in FIG. 18 excluding the 2500 ppm HA preparation to reduce the scale.

As seen in FIGS. 16 through 19, while PEO-1000 demonstrates a pure Newtonian behavior (independence of viscosity on shear rate) at the concentrations as high as 1000 ppm and below, the HA-990 shows a non-Newtonian behavior at the all tested concentrations except 100 ppm. Viscosities were found to be the same for HA-990 at C=100 ppm and PEO-1000 at C=500 ppm.

4. Elasticity Tests.

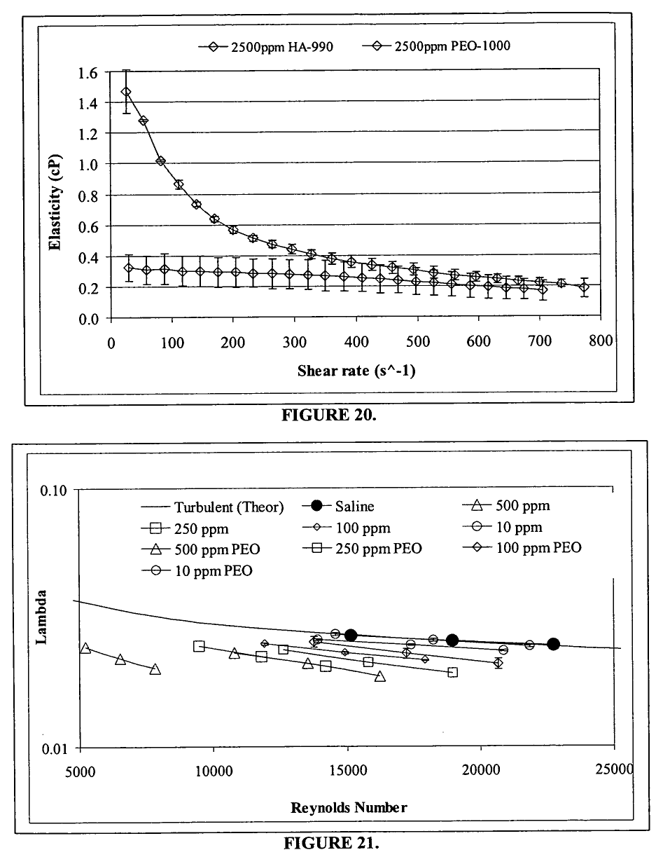

Elasticity of HA-990 2500 ppm solution measured by Vilastic was found to be dependant on shear rates providing additional evidence of a strong non-Newtonian behavior of this solution (FIG. 20). Data on elasticity of a 2500 ppm solution of PEO-1000 is shown in FIG. 20 for a comparison demonstrating much slighter non-Newtonian behavior of this polymer at the same concentration as HA-990. Elasticity of both polymer solutions at 1000 and 500 ppm concentrations were found to be negligible.

III. Hydrodynamic Evaluation.

FIG. 21 shows the results of several tests with turbulent flow of the HA-990 and PEO-1000 in a pipe. The plot shows friction factor (Lambda) versus Reynolds number.

As seen in FIG. 21, at concentrations of 10 ppm flow resistance of the HA-990 is slightly lower than that of saline while flow characteristics of PEO-1000 solutions are the same as those of saline. At a 100 ppm concentration both HA-990 and PEO-1000 showed a slight drag reduction (˜15% and ˜12% respectively). At higher concentrations, HA-990 demonstrates higher drag-reducing ability than PEO-1000 at the studied flow rates. Since the viscosity of HA-990 is much higher than that of PEO-1000, Reynolds numbers corresponding to the same flow rates are much lower for the HA solutions. Therefore, it is not possible to directly compare flow resistance of these polymer solutions at the same flow rate. Linear extrapolation of the obtained data to higher Reynolds numbers showed that HA-990 friction factors are much lower than those of PEO-1000 at the concentrations of 250 (about 10%) and 500 ppm (about 40%). In each test, during the second run at the same flow rates PEO-1000 showed a lower drag-reducing effect than during the first run due to mechanical degradation, while HA-990 demonstrated the same drag-reducing effect during both runs.

IV. Mechanical Degradation Study.

It was previously found that polyethylene oxide while being one of the best drag reducers is extremely fragile and loses its drag-reducing ability when exposed to shear stress due to mechanical degradation. The mechanical degradation of the HA-990 was tested in the circulating loop at constant wall shear stress of 45 N/m² (flow rate ˜4.5 L/min) at two solution concentrations of 100 ppm. The HA-990 polymer was found to be resistant to mechanical degradation with no loss of drag-reducing activity over the five hour test period. PEO-1000 at the solution concentration of 100 ppm tested at the same wall shear stress conditions completely lost its drag-reducing ability in 30 min.

V. Microflow Channel Study of the HA-990 Effect on the Laminar Flow Parameters of Red Blood Cell (RBC) Suspensions.

Previous studies demonstrated that drag-reducing polymers added to a RBC suspension flowing in a microchannel with a diameter less that 0.5 mm redistribute RBCs across the microchannel and attenuate the “plasma-skimming” effect. Attenuation of near-wall plasma layer in a channel with rigid walls is accompanied by an increase in driving pressure/wall shear stress. Thus a comparison of driving pressure vs. flow rate for RBC suspensions with and without DRPs characterizes microhemodynamic properties of polymer preparations.

In the study, farm fresh bovine RBCs were washed with PBS and resuspended in a PBS/albumin solution at a hematocrit of 22.5%. RBC shape was verified using light microscopy. The effects of HA-990 and PEO-1000 additives on the flow of RBCs in a straight microchannel in laminar flow was studied using a circulating system consisting of a syringe pump, a pressure transducer, and a capillary tube with a diameter of 410 mm and length of 12.7 cm. The RBC suspension in the syringe was kept well mixed using a small magnetic stirring bar agitated by a magnet from outside, and the capillary was oriented in a vertical position in order to prevent RBC sedimentation on the walls. Flow rates were varied from 0.5 to 2 ml/min. Pressure vs. flow relationships were measured for each polymer at each concentration (10 ppm and 50 ppm), normalized to account for pressure change due to viscosity alone, and compared to the control RBC suspension in PBS with no polymer added. In such settings, an increase in pressure compared to control would indicate that the RBCs moved closer to the channel walls and near wall viscosity increased.

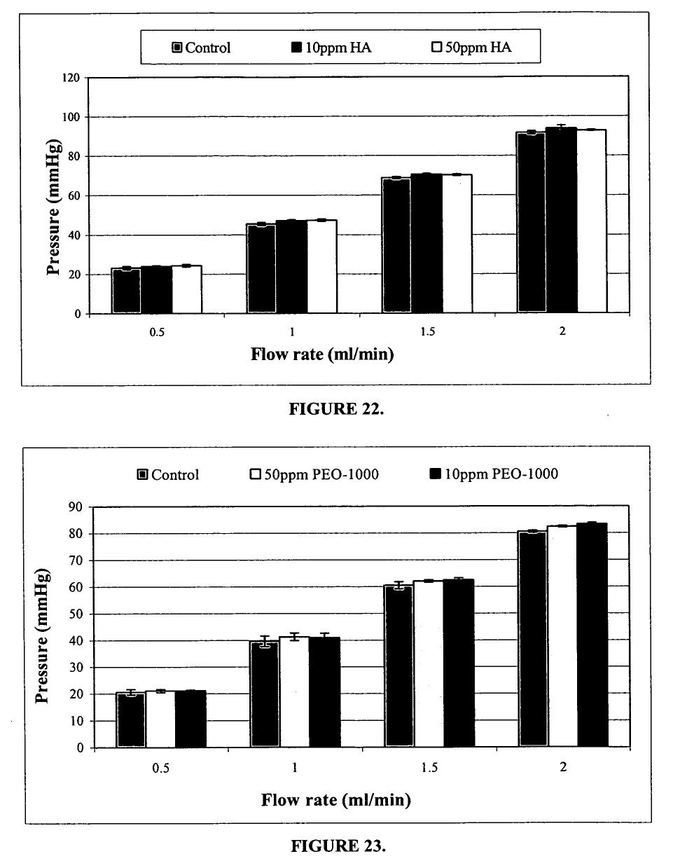

The results of these studies are shown in Tables 2 and 3 and FIGS. 22 and 23. Table 5 shows the percentage of changes in the driving pressure for each flow rate after addition of HA-990 to the RBC suspension. HA produced a slight increase in pressure at both concentrations. Table 6 shows pressure changes due to an addition of 10 and 50 ppm of PEO-1000 to a RBC suspension. The typical increase in driving pressure caused by effective drag-reducers at the studied flow conditions and concentrations was 20-30%.

TABLE 5
% Increase in Pressure

| Flow Rate | Control P | HA 10 ppm P | HA 50 ppm P |
|---|---|---|---|
| (mL/min) | (mmHg) | (mmHg) | (mmHg) |
| 0.5 | 0.0 | 3.1 | 5.1 |
| 1 | 0.0 | 3.8 | 4.1 |
| 1.5 | 0.0 | 2.5 | 2.0 |
| 2 | 0.0 | 2.4 | 1.3 |

TABLE 6
% Increase in Pressure

| Flow Rate | Control | PEO 10 ppm | PEO 50 ppm |
|---|---|---|---|
| (mL/min) | (mmHg) | (mmHg) | (mmHg) |
| 0.5 | 0.0 | 2.1 | 2.1 |
| 1 | 0.0 | 3.2 | 4.2 |
| 1.5 | 0.0 | 3.2 | 2.6 |
| 2 | 0.0 | 3.4 | 2.3 |

FIGS. 22 and 23 show the actual results of the microchannel studies. There is no significant difference between flow of RBC suspensions with and without addition of HA-990 or PEO-1000 at the all studied flow rates.

VI. Microscopic Microchannel Studied of the Effects of Polymer Additives on RBC Distribution Across the Channel

Microscopic studies of bovine RBC suspension (20% Ht in PBS/albumin solution) flow (flow rate=0.2 ml/min) were performed in a microchannel with dimensions: 200 mm×80 mm×50 mm main channel, 50 mm×80 mm×20 mm branch.

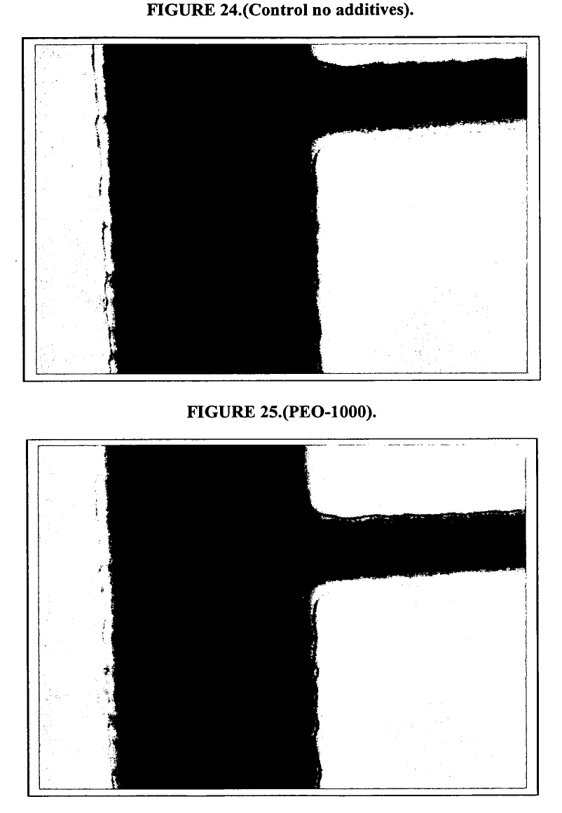

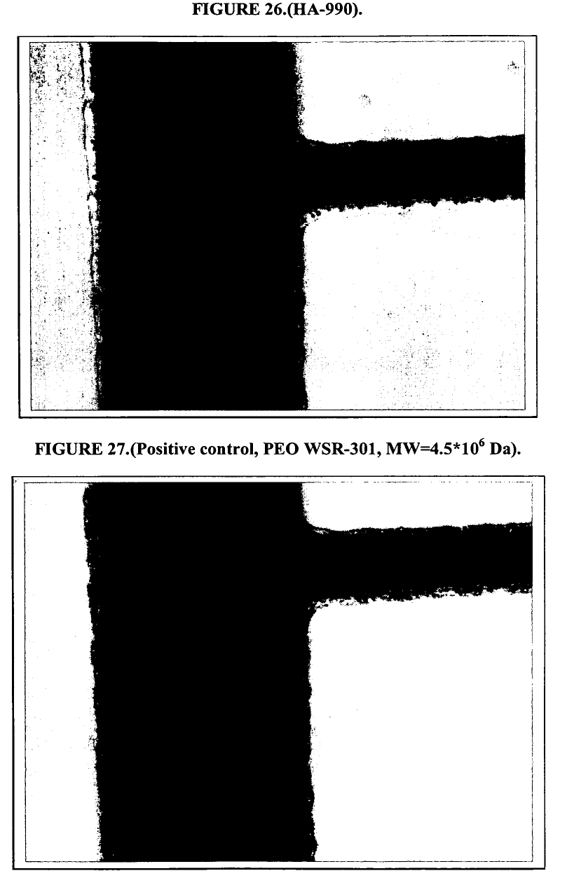

FIGS. 24 through 27 show images obtained in these test with a 10 ppm concentration of the additives specified.

Only the positive control, obtained with an additive of the PEO WSR-301 polymer that has much higher MW, demonstrated a significant increase in the near-wall concentration of RBCs. Control and PEO-1000 samples did not show any RBCs in the near-wall plasma layer. A small amount of RBCs are present in the near-wall space in the RBC suspension with HA-990 added.

The data obtained in this study demonstrated that the HA-990 additive had much better drag-reduction efficiency at concentrations of 250 and 500 ppm than PEO-1000 at the same concentrations. It was found that HA-990 was very stable against mechanical stress. No mechanical degradation was observed after 5 hours of exposure to high flow shear stress which caused very fast total degradation of PEO-1000 (within 30 min). Viscosity and elasticity of HA-990 were found to be much higher that those of PEO-1000 at the same concentrations. In the capillary tube with diameter of 410 μm neither of the polymers shows a significant increase in driving pressure which signified that in this size of microchannels where a near-wall plasma layer was not well developed, these two polymers, HA-990 and PEO-1000, did not change distribution of RBCs across the tube at the studied concentrations. In spite of notable drag-reducing activity at relatively high concentrations in solution, PEO-1000 did not show the ability to affect flow of RBC suspension in a smaller microchannel (200 μm diameter) and thus the ability to change RBC distribution in microvessels. HA-990 demonstrated a slight effect on the RBC flow behavior in this microchannel causing relocation of some RBCs to the near-wall space.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

I claim:

1. A blood soluble drag reducing composition, comprising hyaluronic acid or a physiologically acceptable salt thereof and a pharmaceutically acceptable carrier.

2. The blood soluble drag reducing composition of claim 1, wherein the physiologically acceptable salt of hyaluronic acid is a sodium salt.

3. The blood soluble drag reducing composition of claim 1, wherein the molecular weight of the hyaluronic acid or physiologically acceptable salt is from that of an oligosaccharide to about 7,000 kD.

4. The blood soluble drag reducing composition of claim 3, wherein the physiologically acceptable salt of hyaluronic acid is a sodium salt.

5. The blood soluble drag reducing composition of claim 4, wherein the molecular weight of the sodium salt of hyaluronic acid is between about 600 kD and about 1700 kD.

6. The blood soluble drag reducing composition of claim 5, wherein the concentration of the sodium salt of hyaluronic acid is between about 0.1 ppm and about 1000 ppm in the blood.

7. A method to increase aortic blood flow, increase arterial blood flow, increase capillary blood flow, increase venous blood flow, decrease blood pressure, decrease peripheral vascular resistance, diminish the development of atherosclerosis, or prevent lethality of hemorrhagic shock, comprising the step of administering a therapeutically acceptable amount of hyaluronic acid or a physiologically acceptable salt thereof to a subject in need thereof.

8. The method of claim 7, wherein the physiologically acceptable salt of hyaluronic acid is a sodium salt.

9. The method of claim 7, wherein the molecular weight of the hyaluronic acid or physiologically acceptable salt thereof is from that of an oligosaccharide to about 7,000 kD.

10. The method of claim 9, wherein the physiologically acceptable salt of hyaluronic acid is a sodium salt.

11. The method of claim 10, wherein the molecular weight of the sodium salt of hyaluronic acid is between about 600 kD and about 1700 kD.

12. The method of claim 11, wherein the concentration of the sodium salt of hyaluronic acid is between about 10 ppm and about 1000 ppm in the blood.

13. A method to increase aortic blood flow, increase arterial blood flow, increase capillary blood flow, increase venous blood flow, decrease blood pressure, decrease peripheral vascular resistance, diminish the development of atherosclerosis, or prevent lethality of hemorrhagic shock, comprising the step of administering a therapeutically acceptable pharmaceutical composition comprising hyaluronic acid or a physiologically acceptable salt thereof and a pharmaceutically acceptable carrier to a subject in need thereof.

14. The method of claim 13, wherein the physiologically acceptable salt of hyaluronic acid is a sodium salt.

15. The method of claim 13, wherein the molecular weight of the hyaluronic acid or physiologically acceptable salt thereof is from that of an oligosaccharide to about 7,000 kD.

16. The method of claim 15, wherein the physiologically acceptable salt of hyaluronic acid is a sodium salt.

17. The method of claim 17, wherein the molecular weight of the sodium salt of hyaluronic acid is between about 600 kD and about 1700 kD.

18. The method of claim 11, wherein the concentration of the sodium salt of hyaluronic acid is between about 0.1 ppm and about 1000 ppm in the blood.

19. A packaged pharmaceutical comprising
hyaluronic acid or a physiologically acceptable salt thereof; and
instructions to use said hyaluronic acid or a physiologically acceptable salt thereof to increase aortic blood flow, increase capillary blood flow, increase arterial blood flow, increase venous blood flow, decrease blood pressure, decrease peripheral vascular resistance, diminish the development of atherosclerosis, or prevent lethality of hemorrhagic shock.

20. The packaged pharmaceutical of claim 19, wherein the physiologically acceptable salt of hyaluronic acid is a sodium salt.

21. The packaged pharmaceutical of claim 19, wherein the molecular weight of the hyaluronic acid or physiologically acceptable salt is from that of an oligosaccharide to about 7,000 kD.

22. The packaged pharmaceutical of claim 21, wherein the physiologically acceptable salt of hyaluronic acid is a sodium salt.

23. The packaged pharmaceutical of claim 22, wherein the molecular weight of the sodium salt of hyaluronic acid is between about 600 kD and about 1700 kD.

24. The packaged pharmaceutical of claim 23, wherein the concentration of the sodium salt of hyaluronic acid is between about 0.1 ppm and about 1000 ppm in the blood.

25. A packaged pharmaceutical comprising a pharmaceutical composition comprising:
hyaluronic acid or a physiologically acceptable salt thereof and a pharmaceutically acceptable carrier; and
instructions to use said hyaluronic acid or a physiologically acceptable salt thereof to increase aortic blood flow, increase capillary blood flow, increase arterial blood flow, increase venous blood flow, decrease blood pressure, decrease peripheral vascular resistance, diminish the development of atherosclerosis, or prevent lethality of hemorrhagic shock.

26. The packaged pharmaceutical of claim 25, wherein the physiologically acceptable salt of hyaluronic acid is a sodium salt.

27. The packaged pharmaceutical of claim 25, wherein the molecular weight of the hyaluronic acid or physiologically acceptable salt thereof is from that of an oligosaccharide to about 7,000 kD.

28. The packaged pharmaceutical of claim 27, wherein the physiologically acceptable salt of hyaluronic acid is a sodium salt.

29. The packaged pharmaceutical of claim 28, wherein the molecular weight of the sodium salt of hyaluronic acid is between about 600 kD and about 1700 kD.

30. The packaged pharmaceutical of claim 29, wherein the concentration of the sodium salt of hyaluronic acid is between about 0.1 ppm and about 1000 ppm in the blood.
